(19) United States
(12) Patent Application Publication
(10) Pub. No.: US 2014/0328937 A1
(43) Pub. Date: Nov. 6, 2014

(54) Drug Delivery Systems and Use Thereof

(71) Applicant: Massachusetts Eye and Ear Infirmary, Boston, MA (US)

(72) Inventors: Karen G. Carrasquillo, Cambridge, MA (US); Anthony P. Adamis, Jamaica Plain, MA (US); Joan W. Miller, Winchester, MA (US); Evangelos S. Gragoudas, Lexington, MA (US)

(73) Assignee: Massachusetts Eye and Ear Infirmary, Boston, MA (US)

(21) Appl. No.: 14/198,546

(22) Filed: Mar. 5, 2014

Related U.S. Application Data
(63) Continuation of application No. 13/753,124, filed on Jan. 29, 2013, now abandoned.
Application No. 13/753,124 is a continuation of application No. 13/247,458, filed on Sep. 28, 2011, now abandoned.
Application No. 13/247,458 is a continuation of application No. 12/753,507, filed on Apr. 2, 2010, now abandoned.
Application No. 12/753,507 is a continuation of application No. 10/979,785, filed on Nov. 2, 2004, now abandoned.
Application No. 10/979,785 is a continuation-in-part of application No. PCT/US03/04645, filed on Feb. 17, 2003.
Application No. PCT/US03/04645 is a continuation-in-part of application No. 10/139,656, filed on May 2, 2002, now Pat. No. 7,563,255.
(60) Provisional application No. 60/438,651, filed on Jan. 8, 2003.

(51) Int. Cl. A61K 9/00; A61K 9/16; B01J 14/00; A61K 31/7088

(52) U.S. Cl. CPC A61K 9/0048; A61K 31/7088; A61K 9/16; B01J 14/00
USPC 424/501; 514/44 R; 264/11

(57) ABSTRACT

The invention provides a microsphere formulation for the sustained delivery of an aptamer, for example, an anti-Vascular Endothelial Growth Factor aptamer, to a preselected locus in a mammal, such as the eye. In addition, the invention provides methods for making such formulations, and methods of using such formulations to deliver an aptamer to a preselected locus in a mammal. In particular, the invention provides a method for delivering the aptamer to an eye for the treatment of an ocular disorder, for example, age-related macular degeneration.

### RELATED APPLICATIONS

This application is a continuation-in-part of International Application Ho, PCT/US03/03645, filed on Feb. 17, 2003, and published in English, and is a continuation-in-part of U.S. Ser. No. 10/139,656, filed May 2, 2002, and claims the benefit of and priority to U.S. provisional application 60/438,651, filed Jan. 8, 2003, the disclosures of which are hereby incorporated by reference.

### FEDERAL FUNDING

The invention was made with funds from the National Eye Institute Grants EY12611 and EY11627. The government has certain rights in the invention.

### FIELD OF THE INVENTION

The invention relates to methods and compositions for delivering a Vascular Endothelial Growth Factor inhibitor to a mammal and more particularly to methods and compositions for delivering an anti-Vascular Endothelial Growth Factor aptamer to a mammal.

### BACKGROUND OF THE INVENTION

The way a particular drug is administered to a recipient can significantly affect the efficacy of the drug. For example, some therapies, in order to be optimal, require that the drug be administered locally to a particular target site. Furthermore, some of those drugs need to be present at the target site for a prolonged period of time to exert maximal effect.

One approach for achieving localized drug delivery involves the injection of drug directly into the site of desired drug activity. Unfortunately, this approach may require periodic injections of drug to maintain an effective drug concentration at the target site. In order to prolong the existence at the target site, the drug may be formulated into a slow release formulation (see, for example, Langer (1998) NATURE 392, Supp. 5-10). Following administration, drug then is released via diffusion out of, or via erosion of the matrices. Alternatively, drug can be encapsulated within a semi-permeable membrane or liposome. Following administration, the drug is released either by diffusion through the membrane or via breakdown of the membrane. However, problems associated with localized drug injection can include, for example, repeated visits to a health care professional for repeated injections, difficulty in stabilizing drugs within slow release formulations, and the control of the concentration profile of the drug over time at the target site.

Another approach for localized drug-delivery includes the insertion of a catheter to direct the drug to the desired target location. The drug cart be pushed along the catheter from a drug reservoir to the target site via, for example, a pump or gravity feed. Typically, this approach employs an extracorporeal pump, an-extracorporeal drug reservoir, or both an extracorporeal pump and extracorporeal drug reservoir. Disadvantages can include, for example, the risk of infection at the catheter's point of entry into the recipient's body, and that, because of their size, the pump and/or the reservoir may compromise the mobility and life style of the recipient.

Over the years, implantable drug delivery devices have been developed to address some of the disadvantages associated with localized injection of drug or the catheter-based procedures. While a variety of implantable drug delivery devices have been developed to date, there is still an ongoing need in the art for reliable drug delivery systems that permit the localized delivery of a drug of interest over a prolonged period of time.

### SUMMARY OF THE INVENTION

The invention is based, in part upon the discovery that a anti-Vascular Endothelial Growth Factor (VEGF) aptamer, when encapsulated in a biocompatible polymer microsphere can be released under physiological conditions over a period of least 20 days, and that the aptamer, when released, retains its biological activity.

In one aspect, the invention provides microspheres for the sustained release of an anti-VEGF aptamer. The microspheres include the anti-VEGF aptamer and a biocompatible polymers. where the amount of the aptamer in the microsphere varies from 0.1% to 30% (w/w) (e.g., 0.1%, 1%, 10%, 20%, or 30% (w/w)), 0.1% to 10% (w/w) (e.g., 0.5%, 2%, or 5% (w/w)), or, desirably, 0.5% to 5% (w/w) (e.g., 0.5%, 1.0%, 1.5%, 2.0%, 2.5%, 3%, 3.5%, 4% or 4.5% (w/w)) of the microsphere. The microspheres may further include a stabilizer, for example, a sugar, for example, trehalose. The mass ratio of aptamer to trehalose in the microsphere is at least 1:1, 1:2, 1:3, 1:4, or 1:5. A mass ratio of aptamer to trehalose in the microsphere of at least 1:3 is preferred.

In one embodiment, the biocompatible polymer is a degradable polymer. Degradable polymers useful in the preparation of the microspheres include polycarbonate, polyanhydride, polyamide, polyester, polyorthoester, and copolymers or mixtures thereof. Exemplary polyesters include poly(lactic acid), poly(glycolic acid), poly(lactic acid-co-glycolic acid), polycaprolactone, blends thereof and copolymers thereof. Desirably, the half-life for the degradation of the degradable polymer under physiological conditions is at least about 20 days and more preferably is at least about 30 days. In one embodiment, the microspheres comprise a poly(lactic acid co-glycolic acid) (PLGA) polymer.

In another embodiment, the biocompatible polymer is a non-degradable polymer. Non-degradable polymers useful in the preparation of the microspheres include polyether, vinyl polymer, polyurethane, cellulose-based polymers, and polysiloxane. Exemplarly polyethers include poly (ethylene oxide), poly (ethylene glycol), and poly (tetramethylene oxide). Exemplary vinyl polymers include polyacrylates, acrylic acids, poly (vinyl alcohol), poly (vinyl pyrolidone), and poly (vinyl acetate). Exemplary cellulose-based polymers include cellulose, alkyl cellulose, hydroxyalkyl cellulose, cellulose ether, cellulose ester, nitrocellulose, and cellulose acetate.

Whichever biocompatible polymer is used, in one embodiment, the microspheres preferably have an average diameter in the range from about 1 μm to about 200 μm (e.g., 10, 25, 50, 75,100, 125, 150, 175, or 200 μm), from about 5 μm to about 100 μm (e.g., 10, 20, 30, 40, 50, 60, 70, 80, 90, or 100 μm), and from about 10 μm to about 50 μm (e.g., 12.5, 25, 35, or 45 μm). In one embodiment, the microspheres have an average diameter of about 15 μm.

In another aspect the invention provides a method of preventing, treating or inhibiting an ocular disease state in a mammal in need thereof using any of the microsphere compositions described herein. The method includes administering the microspheres to a mammal in an amount sufficient to treat or inhibit the disease. The microspheres can be administered, for example, via intravitreal injection or via transcleral delivery. In the transcleral delivery approach, the microspheres are disposed upon the outer surface of the sclera. In such a system, once the aptamer is released out of the microsphere, the aptamer traverses the sclera to exert its effect, for example, reduce or inhibit the activity of the native VEGF molecule and/or the cognate VEGF receptor, within the eye.

The microspheres may be used to treat a variety of ocular disorders including, for example, optic disc neovascularization, iris neovascularization, retinal neovascularization, choroidal neovascularization, corneal neovascularization, vitreal neovascularization, glaucoma, pannus, pterygium, macular edema, vascular retinopathy, retinal degeneration, uveitis, inflammatory diseases of the retina, and proliferative vitreotetinopathy. The corneal neovascularization to be treated or inhibited may be caused by trauma, chemical burns and corneal transplantation. The iris neovascularization to be treated or inhibited may he associated with diabetic retinopathy, vein occlusion, ocular tumor and retinal detachment. The retinal neovascularization to be treated or inhibited may be associated with diabetic retinopathy, vein occlusion, sickle cell retinopathy, retinopathy, of prematurity, retinal detachment, ocular ischemia and trauma. The intravitreal neovascularization to be treated or inhibited may be associated with diabetic retinopathy, vein occlusion, sickle cell retinopathy, retinopathy of prematurity, retinal detachment, ocular ischemia and trauma. The choroidal neovascularization to be treated or inhibited may be associated with retinal or subretinal disorders, such as, age-related macular degeneration, presumed ocular histoplasmosis syndrome, myopic degeneration, angioid streaks and ocular trauma.

In another aspect, the invention provides a method of preparing the microspheres. The method includes the steps of: (a) dissolving a biocompatible polymer in a solvent to form a solution; (b) combining an aptamer of interest with the solution to produce a mixture; (c) optionally combining the mixture of step (b) with a coacervating agent (optionally, while homogenizing the solution); and (d) permitting the biocompatible polymer to form microspheres containing the aptamer. During step (b), a stabilizer, for example, a sugar, for example, trehalose may be added to the mixture. For example, when trehalose is added, the mass ratio of aptamer to trehalose preferably is at least 1:3.

### BRIEF DESCRIPTION OF THE DRAWINGS

The foregoing and other aspects of the invention and the various features thereof may be more fully understood from the following description when read together with the accompanying drawings, in which:

FIG. 1 is a schematic illustration in perspective view of an exemplary transcleral drug delivery device employing a drum-type drug-containing reservoir and a rotatable puncturing member.

FIGS. 2A-2B are scanning electron micrographs of PLGA microspheres. FIG. 2A depicts PLGA microspheres loaded with an anti-VEGF aptamer EYE001 before incubation with release medium. FIG. 2B depicts the microspheres after 10 days of exposure to aqueous release medium. The average diameter of the microspheres was 14±6 μm, based upon the average diameter of approximately 30 different microspheres.

FIGS. 3A-3B depict the release profiles of excipient-free EYE001 and EYE001 colyophilized with trehalose. FIG. 3A depicts the cumulative release profile of (square) excipient-free EYE001 and (triangle) EYE001-Trehalose (Tre) from PLGA microspheres. FIG. 3B depicts the correlation between the amount of EYE001 released and the square root of time for (square) excipient-free EYE001 and (triangle) EYE001-Tre. The correlation coefficients of the released formulations were 0.9852 and 0.9946, respectively.

FIG. 4 is a graph depicting the circular dichroism (CD) spectra of reconstituted EYE001 formulations under different lyophilization conditions. The spectra were recorded after reconstitution of samples in phosphate buffered saline.

FIGS. 5A-5C are graphs depicting the results of cell proliferation assays of human umbilical vein endothelial cells (HUVEC) incubated with EYE001 formulations after release from PLGA microspheres. Each graph indicates the incubation condition and the time-point at which EYE001 was collected after release from PLGA. The mean values represent the average cell count results for each condition in three independent experiments. FIGS. 5A, 5B, and 5C represent short-, mid-, and long-term release time points, respectively (*P>0.05; #P>0.05 versus VEGF-induced cell proliferation; n=3 for all time points). In each chart, the number of hours the aptamer had been released from the microspheres is indicated (FIG. 5A, for example, shows the activity of aptamer having being released from the microspheres for 24, 48 and 72 hours).

FIG. 6 is a schematic illustration of an exemplary device for measuring transcleral drug delivery.

FIGS. 7A-7C depict scanning electron microscope images of bare rabbit sclera (FIG. 7A), rabbit sclera (orbital surface) exposed to PLGA microspheres for 18 hours (FIG. 7B) and rabbit sclera (orbital surface) exposed to PLGA microsphere for 6 days (FIG. 7C).

FIG. 8 is a bar chart showing that blood vessel leakage is reduced in eyes treated with an anti-VEGF aptamer (EYE001) relative to leakage in control eyes without EYE001 treatment. The values presented are the average result of 6 different experiments (n=6). The standard error =*/* where * is the standard deviation of the original distribution and *n is the square root of the sample size.

### DETAILED DESCRIPTION OF THE INVENTION

The invention provides a composition of matter that permits the sustained delivery of aptamers to a preselected locus in a mammal. The aptamers, preferably are anti-VEGF aptamers. The aptamers, for example, the anti-VEGF aptamers, may be used in the treatment of a variety of disorders associated with VEGF activity, for example, neovasculature associated with the activation of the VEGF receptor by a VEGF molecule. In such a system, the administration of the anti-VEGF aptamer can bind to a nucleic VEGF molecule thereby preventing it from binding to its cognate VEGF receptor. The aptamers may be useful in the treatment of ocular disorders that are initiated, mediated or facilitated by means of the VEGF receptor. The microspheres permit, the sustained release of the aptamers to the site of interest so that the aptamers can exert their biological activity over a prolonged period of time.

Once implanted, the aptamer containing microspheres may deliver the aptamer of interest over a prolonged period of time into the tissue or body fluid surrounding the microspheres thereby imparting a localized prophylactic and/or therapeutic effect it is contemplated that the microspheres may administer the aptamer of interest over a period of weeks (for example, 1, 2, or 3 weeks), and more preferably months (for example, 1, 2, 3, 4, 5, 6, 7, 8, 9, 10, or 11 months), or longer.

1Aptamers

Aptamers are chemically synthesized oligonucleotides that adopt highly specific three-dimensional conformations. Large numbers of different aptamers can be synthesized using the Systematic Evolution of Ligands of Exponential enrichment (SELEX) process, which is a combinatorial chemistry method they allows for the identification of specific sequences that bind to a target of interest. The properties of aptamers can be refined by negative and/or positive selection methods to identify; for example, aptamers that bind to their desired target, but do not bind to other related targets.

Nucleic acids (e.g., RNA, DNA and mixed RNA-DNA molecules) may be prepared as oligonucleotides. These oligonucleotide sequences, preferably, are 6, 8, 10, 11, 12, 13, 14, 15, 16, 17, 18, 19, 20, 21, 22, 23, 24 or 25 nucleotides in length, but may be as long as 40, 50, 75, or 100 nucleotides in length. In general, a minimum of 6 nucleotides, preferably 10 nucleotides, more preferably 14 to 20 nucleotides, is necessary to effect specific binding. In general, the oligonucleotides are preferably single-stranded (ss) DNA molecules, but may be double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). Chemical modifications that enhance an aptamers specificity or stability are preferred.

Although aptamers may contain unmodified nucleotides, it is contemplated that one or more of the nucleotides in the aptamer may be modified so as to modulate binding specificity, stability, and/or longevity of the resulting aptamer. Chemical modifications that may be incorporated into aptamers, .and other nucleic acids include, without limitation, base modifications, sugar modifications, and backbone modifications. The base residues in aptamers may be other than naturally occurring bases (e.g., A, G, C, T, U, 5MC, and the like). Derivatives of purines and pyrimidines are known in the art (e.g. aziridinylcytosine, 4-acetyleytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methladenine, 2-methylguanine, 3-methylcytosine, 5-methlcytosine (5MC), N6-methyladenine, 7-methylguanine, 5-methylaminomethyluracil, 5-methoxyaminomethyl-2-thioaracil, beta-D-mannosylqueosine, 5-methoxyurcil, 2-methylthio-N-6-isopentenylade-nine, uracil-5-oxyacetic acid methylester, pseadoaracil, queosine, 2-thiocytosine, 5-methyl-2-thiouracil, 2-thiouracil, 4-thiouracil, 5-methyluracil, uracil-5-oxyacetic acid, and 2,6-diamiopurine). In addition to nucleic acids that incorporate one or more of such base derivatives, nucleic acids having nucleotide residues that are devoid of a purine or a pyriroidine base may also be included in aptamers.

The sugar residues in aptamers may be other than conventional ribose and deoxyribose residues. By way of non-limiting example, substitution at the 2′-position of the furanose residue can enhance nuclease stability. An exemplary, but not exhaustive list, of modified sugar residues includes 2′ substituted sugars such as 2′-O-methyl, 2′-O-alkyl, 2′-O-allyl, 2′-S-alkyl, 2-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, feranose sugars, sedoheptuloses, acyclic analogs and abasic nucleoside analogs such as methyl riboside, ethyl riboside or propylriboside.

Chemically modified backbones include, for example, phosphorothioates, chiral phosphorothioates, phosphorodithiotes, phosphotriesters, aminoalkylphosphotriesters, methyl and other alkyl phosphonafes including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranephosphates having normal 3′-5′ linkages; 2′-5′linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpliolino linkages; siloxane backbones; sulfide, sulfoxide and sulfone backbones; formacetyl and thioformacetyl backbones; methylene formaectyl and thioformaectyl backbones; alkene containing backbones; salfamate backbones; methyleneimino and methylenehydrazino backbones; sulfonate and sulfonamide backbones; and amide backbones.

Aptamers are delivered to a preferred site of a subject (e.g., a mammal, such as a human) using microspheres of the invention. In one preferred embodiment, the microspheres of the invention permit the sustained delivery of an anti-VEGF aptamer. An anti-EVGF aptamer is a nucleic acid molecule capable of binding specifically to a native VEGF molecule and/or a native VEGF receptor under physiological conditions and reducing or eliminating the biological activity of the VEGF molecule or the VEGF receptor. For example, the VEGF aptamer can bind to a native VEGF molecule thereby reducing the ability of the VEGF to bind to its cognate VEGF receptor agenting the VEGF molecule from binding to its cognate VEGF receptor. Accordingly, the VEGF aptamer modulates the activity (for example, prevents activation) of the VEGF receptor. One anti-VEGF aptamer of interest is known in the art as EYE001 and was formerly known in the art as NX1838 (see, Drolet et al. (2000) PHARM. RES. 17:1503-1510; Ruckman et al. (1998) J. BIOL. CHEM. 273:20556-20567; Carrasquillo et al. (2003) INVEST. OPHTMAL. VIS. SCI. 44:290-299). EYE001 is available from Eyetech Pharmaceuticals (New York, N.Y.) and was identified by the systematic evolution of ligands by expomential enrichment (SELEX) process (Ruckman et al. (1998) J. BIOL. CHEM. 273:20556-20567; Costantino et al. (1998) J. PHARM. SCI. 87:1412-1420). EYE001 can be supplied as a liquid formulation of 3 mg/200 μL saline solution.

EYE001 is a pegylated RNA aptamer of 50 kDa, with an A-type secondary structure, 40 mg/mL solubility, and a net negative charge of −29. The structure of EYE001 is as follows:

5′-[40 kd PEG]-[HN-(CH₂)₅O]-pC_{f}pGₘpGₘpAᵣpAᵣpU_{f}pC_{f}pAₘpGₘ
pU_{f}pGₘpAₘpAₘpU_{f}pGₘpc_{f}pU_{f}pU_{f}pAₘpU_{f}pAₘpC_{f}pAₘpU_{f}pC_{f}pC_{f}pGₘ
3′-p-3′dT.

The 40 kd PEG component represents two 20 kilodalton-poly(ethylene glycol) polymer chains covalently attached to the two amine groups on a lysine residue via carbamate linkages. This moiety is in turn linked to the oligonucleotide via a bifunctional, amino linker, [HN—(CH₂)₅O—]. The linker is attached to the oligonucleotide by a standard phosphodiester bond; p represents the phosphodiester functional groups that link sequential nucleosides and that link the amino linker to the oligonucleotide. All of the phosphodiester groups are negatively charged at neutral pH and have a sodium atom as the counter ion; Gₘ or Aₘ and C_{f} or U_{f} and Aᵣ represent 2′-methoxy, 2′-fluoro and 2′-hydroxy variations of their respective purines and pyrimidines; C, A, U, and G is the single letter code for cytidylic, adenylic, uridylic, and guanylic acids. All phosphodiester linkages of this compound, with the exception, of the 3′-terminus, connect the 5′ and 3′ oxygens of the ribose ring. As shown, the phosphodiester linkage between the 3′-terminal dT and the penultimate Gₘ links their respective 3′-oxygens. This is referred to as a 3′,3′ cap.

Although the EYE001 aptamer is currently preferred, it is contemplated that the microspheres of the invention may deliver other aptamers of interest of a sustained basis.

2. Aptamer Containing Microspheres and Fabrication Thereof

In order to permit sustained delivery of an aptamer of interest, the aptamer is encapsulated within microsphere comprising a biocompatible polymer. The choice of the appropriate microsphere system will depend upon rate of aptamer release required by a particular regime. The aptamer may be homogeneously or heterogeneously distributed within the microspheres. Furthermore, both non-degradable and degradable microspheres can be used. Suitable microspheres may include polymers and polymeric matrices, non-polymeric matrices, or inorganic and organic exciptents and diluents such as, but not limited to, calcium carbonate and sugar. Synthetic polymers are preferred because generally they are more reliable, more reproducible and produce more defined release profiles. The microspheres can be designed so that aptamers having different molecular weights are released by diffusion through or degradation of the microspheres.

As mentioned above, it is contemplated that useful biocompatible polymers may include biodegradable and/or non-biodegradable polymers. Suitable biodegradable polymers useful in the preparation of the microspheres include polycarbonates, polyanhydrides, polyamides, polyesters, polyorthoesters, and copolymers or mixtures thereof. Exemplary polyesters include poly(lactic acid), poly(glycolic acid), poly(laetic acid-co-glycolic acid), polycapralactone, blends thereof and copolymers thereof. Desirably, the half-life for the degradation of the degradable polymer under physiological conditions is at least about 20 days and more preferably is at least about 30 days. Suitable non-biodegradable polymers useful in the preparation of microspheres include pelyethers, vinyl polymers, polyurethanes, cellulose-based polymers, and polysiloxanes. Exemplary polyethers include poly (ethylene oxide), poly (ethylene glycol), and poly (tetramethylene oxide). Exemplary vinyl polymers include polyacrylates, acrylic acids, poly (vinyl alcohol), poly (vinyl pyrolidone), and poly (vinyl acetate). Exemplary cellulose-based polymers include cellulose, alkyl cellulose, hydroxyalkyl cellulose, cellulose ethers, cellulose esters, nitrocellulose, and cellulose acetates.

It is contemplated that in order to produce the appropriate release kinetics, the microspheres may comprise one or more biodegradable polymers or one or more non-biodegradable polymers. Furthermore, it is contemplated that the microspheres may comprise one or more biodegradable polymers in combination with one or more non-biodegradable polymers. Whichever biocompatible polymer is used, in one embodiment, the microspheres preferably have an average diameter in the range from about 1 μm to about 200 μm (e.g., 10, 25, 50, 75, 100, 125, 150, 175, or 200 μm), from about 5 μm to about 100 μm (e.g., 10, 20, 30, 40, 50, 60, 70, 80, 90, or 100 μm), and from about 10 μto about 50 μm (e.g., 12.5, 25, 35, or 45 μm). In one embodiment, the microspheres have an average diameter of about 15 μm.

Methods for modifying the release parameters of the microsphere are known in the art, and are described, for example, by Martinez-Saucho et al. (2004) INT. J. PHARMACEUTICS 273:45-36). Optimization methods include, for example, varying the polymers and/or polymer ratios to alter the release parameters. In one embodiment, a 1:1 ratio of polycaprolactone to poly(lactic acid) can be used. In another embodiment, a 2:1 ratio of polyeaprolactone to poly(lactic acid) can be used varying ratios of increasing polycaprolactone to poly(lactic acid) content. In other embodiments, the ratios can be varied to increase the molecular weight of the monomer and/or increase the hydrophobicity of the mixture. Preferably, these parameters are altered to allow for the release of aptamer front the mixture for at least one months, two months, three months, or longer.

In a preferred embodiment, the microspheres are fabricated from poly(lactic acid-co-glycolic acid (PLGA). Aptamer containing PLGA microspheres can be prepared, for example, using non-aqueous oil-in-oil methods (see, Carrasquillo et al. (2001) J. CONTROL RELEASE 76:199-208). Briefly, 25 to 31 mg of solid aptamer is suspended in a solution of 200 mg/2 mL PLGA (Resomer 502 H, i.v. (inherent viscosity) 0.16-0.24 dL/g, 0.1% in chloroform, 25° C., molecular weight [Mw] 10 to 12 kDa, half-life for degradation approximately 1 to 1.5 months; Boehringer Ingelheim Pharma KG, Ingelheim, Germany) in methylene chloride using a homogenizer (Polytran, model PT 1200C; Brinkman, Westbury, N.Y.) having a standard 12-mm diameter generator at approximately 20,000 rpm for 1 minute. After suspension of the aptamer, a coacervating agent, for example, poly(dimethylsiloxane), optionally can be added at a rate of 2 mL/min under constant homogenization, to ensure homogeneous dispersion of the coacervating agent, phase separation of PLGA dissolved in methylene chloride, and formation of microspheres. The coacervatiag mixture containing the microspheres then is poured into an Erlenmeyer flask containing 50 mL heptane under constant agitation and stirred for 3 hours at room temperature to allow for hardening of the microspheres. Microspheres then are collected by filtration with the use of a 0.22-μm nylon filter, washed twice with heptane, and dried for 24 hours at a vacuum of 80 mbar.

Encapsulation efficiency can be determined using standard methodologies (Carrasquillo et al. (2001) J. PHARM PHARMACOL. 53:115-120). For example, ten milligrams of PLGA microspheres are placed in 2 mL methylene chloride and stirred for 30 minutes to dissolve the polymer. The solution then is centrifuged at 10,000 rpm for 10 minutes to precipitate the insoluble RNA aptamer. The supernatant then is removed, and the remaining methylene chloride allowed to evaporate. In order to ensure evaporation of the methylene chloride, the sample can be placed in a vacuum for 24 hours. The aptamer then is dissolved in Dulbecco's phosphate-buffered saline (DPBS; GibeoBRL, Grand Island, N.Y.), and the concentration of entrapped aptamer in PLGA determined spectrophotometrically. The percentage encapsulation efficiency can be calculated by relating the experimental aptamer entrapment to the theoretical aptamer entrapment: (experimental/theoretical)×100.

In one embodiment, the microspheres include the anti-VEGF aptamer and a biocompatible polymer, where the amount of the aptamer in the microsphere varies from 0.1% to 30% (w/w) (e.g., 0.1%, 1%, 10%, 20%, or 30% (w/w)), 0.1%: to 10% (w/w) (e.g., 0.5%, 2%, or 5% (w/w)), or, desirably, 0.5% to 5% (w/w) (e.g., 0.5%, 1.0%, 1.5%, 2.0%, 2.5%, 3%, 3.5%, 4% or 4.5% (w/w)) of the microsphere. It is understood that nucleic acids may suffer from depurination and become susceptible to free radical oxidation in aqueous solutions (Lindahl (1993) NATURE 362:709-715; Demple et al. (1994) ANNU REV BIOCHEM. 63:915-948). This effect may be reduced, minimized or eliminated by the addition of a stabilizer, for example, a sugar. An effective stabilizer is the sugar, trehalose. In one embodiment, the mass ratio of aptamer to trehalose in the microsphere is at least 1:3.

It is contemplated that the microspheres may comprise an anti-VEGF aptamer in combination with another angiogenesis inhibitor, that is, a compound that reduces or inhibits the formation of new blood vessels in a mammal. For example, the microspheres may compose two or more different anti-angiogenesis aptamers. Alternatively, the microspheres in addition to containing an anti-VEGF aptamer may also include another type of angiogenesis inhibitor, for example, an angiogenic steroid, for example, hydrocortisone and anecortave acetate (Penn et al. (2000) INVEST. OPHTHALMOL. VIS. SCI. 42:283-290), or another small molecule, for example, thalidomide (D'Amato et al. (1994) PROG. NATL. ACAD. SCI. USA 91:4082-4085).

3. Microsphere Delivery

Once fabricated, the microspheres can be delivered using a variety of delivery devices know in the art. The choice of a particular delivery system will depend upon a variety of factors including, for example, the amount of aptamer that needs to be administered to an individual to exert, an effect, the duration of the microspaces, and the aptamer in the recipient, and the length of time that is needed to treat a particular disorder.

It is contemplated that the aptamer containing microspheres may be used in a variety of different applications. In one embodiment, the microspheres may he used to administer the aptamers to an eye thereby to treat or ameliorate the symptoms of one or more ocular disorders. For examples the microspheres may be particularly useful in the treatment of a variety of ocular disorders, for example, optic disc neovascularization, iris neovascularization, retinal neovascularization, choroidal neovascularization, corneal neovascularization, vitreal neovascularization, glaucoma, pannus, pterygium, macular edema, vascular retinopathy, retinal degeneration, uveitis, inflammatory diseases of the retina, and proliferative vitreoretinopathy. The corneal neovascularization to be treated or inhibited may be caused by trauma, chemical burns and corneal transplantation. The iris neovascularization to be treated or inhibited may be associated with diabetic retinopathy, vein occlusion, ocular tumor and retinal detachment. The retinal neovascularization to be treated or inhibited may be associated with diabetic retinopathy, vein occlusion, sickle cell retinopathy, retinopathy of prematurity, retinal detachment, ocular ischemia and trauma. The intravitreal neovascularization to be treated or inhibited may be associated with diabetic retinopathy, vein occlusion, sickle cell retinopathy, retinopathy of prematurity, retinal detachment, ocular ischemia and trauma. The choroidal neovascularization to be treated or inhibited may be associated with retinal or subretinal disorders of age-related macular degeneration, presumed ocular histoplasmosis syndrome, myopic degeneration, angioid streaks and ocular trauma.

Virtually any method of delivering a medication to the eye may be used for the delivery of microspheres of the invention. In one approach the microspheres can be administered intravitreally, for example, via intravitreal injection. In another approach, the microspheres can be administered transclerally.

With regard to the intravitreal injection approach, methods for optimizing microsphere drug delivery are known in the art (e.g., Martinez-Sancho et al. (2003) J. MICROENCAPSULATION 20:799-810; Martinez-Sancho et al. (2004) INT. J. PHARM. 273:45-56; Khoobeehi et al. (1991) OPHTHALMIC SURG. 22:175-80). In general, intravitreal injection involves loading a glass syringe with microspheres suspended in a buffer, such as hyaluronic acid, and injecting the contents of the syringe through the pars plana of the eye and into the vitreal cavity. This provides a highly effective mechanism for the local delivery and extended release of aptamers from the microspheres.

With regard to transcleral drug delivery, it has been found that certain drugs, when applied to the outer surface of an eye, can traverse the sclera and enter the interior of the eye (see, PCT/US00/00207 and Ambati et al. (2000) INVEST. OPHTHLAM. SCI. 41:1181-1185). More specifically, it has been found that large molecules, for example, immunoglobulin G can diffuse across the sclera of rabbit eyes in a manner consistent with porous diffusion through a fiber matrix (Ambati et al. (2000) supra). This observation has led to the possibility of delivering immunoglobulins and other large compounds transclerally to treat disorders associated with, for example, the retina and choroid (Ambati et al. (2000) supra).

A variety of drug delivery devices may be used to deliver aptamer containing microspheres to the scleral surface of an eye. The microspheres degrade releasing the aptamer, which then traverses the sclera to exert its effect within the eye. Exemplary transcleral drug delivery devices include passive drug delivery devices where drug is released gradually from an implanted device over time (see, for example, U.S. Pat. Nos. 5,300,114; 5,836,935; 6,001,386; and 6,413,540; and International Application No. PCT/US00/28187).

Implantable drug delivery devices that can deliver the microspheres to the surface of the eye include osmotically driven devices. Such devices are available commercially from Durect Corp. (Cupertino, Calif.) under the tradename DUROS®, and from ALZA Scientific Products (Mountain View, Calif.), under the tradename ALZET®. In some devices, the influx of fluid into the device causes an osmotically active agent to swell. The swelling action is employed to push drug from a reservoir out of the device. DUROS® pumps are reported to deliver up to 200 mg of drug at rates as low as 0.5 μL per day. A variety of different osmotically driven drug delivery devices are described, for example, in U.S. Pat. Nos. 4,957,494, 5,236,689 and 5,391,381.

U.S. Pat. Nos. 5,797,898 and 6,123,861 disclose microchip-based drug delivery Devices. A plurality of drug reservoirs are etched into a substrate, for example, a single microchip. Drugs then are sealed within each of the reservoirs with a seal. The seal can be either a material that degrades overtime or a material that dissolves upon application of an electric potential. See also Santini et al. (1999) NATURE 397:335-338, which similarly discloses a solid-state silicon microchip that provides controlled release of a drug of interest via electrochemical dissolution of a thin membrane covering a micro-reservoir filled with drug. Drug delivery may also be accomplished using a flexible microchip device suitable for ophthalmic use. In one embodiment, an implanted microchip device provides for the accurate and controlled local delivery of medication to the eye on a periodic basis for an extended period of time. Suitable microchip devices are known to the skilled artisan and are described, for sample, in U.S. Patent Application No. 20020099359.

Another suitable drug delivery device is described in U.S. Patent Application Publication No. 20030069560 and in International Application No. PCT/US02/14279, which describe a miniaturized, implantable drug delivery device capable of delivering one or more drugs at defined rates to a particular target location over a prolonged period of time. In view of its small size, the drug delivery device is implanted using minimally invasive procedures into a small body cavity (e.g., an eye socket), where it delivers one or more drugs over a prolonged period of time to tissue or body fluid surrounding the implanted device. In one embodiment, the drug delivery device is adapted for attachment to an outer surface of an eye. When attached, the device delivers drug to the surface of the eye, which then passes through the sclera and into the target tissue to ameliorate the symptoms of an ocular disorder.

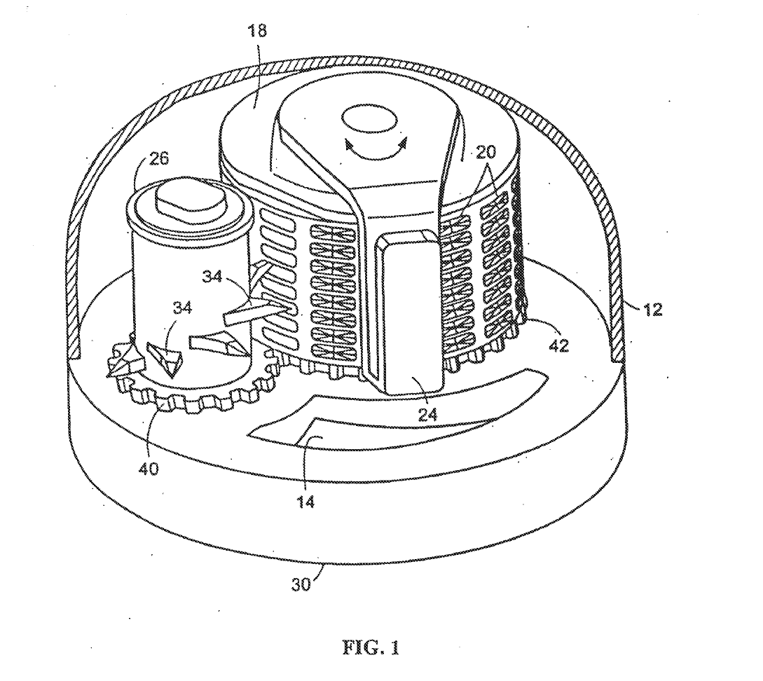

FIG. 1 shows a perspective view of an exemplary electromechanical drug delivery device, as disclosed in International Application No. PCT/US02/14279. In this device, casing 12 (cut-away to reveal the inner components) defines an aperture port 14 and an optional eye contacting surface 30. In this embodiment, the rotational axes of reservoir member 18 and puncturing member 26 are disposed along a plane perpendicular to the plane defined substantially by the eye contacting surface 30. Reservoir member 18, in the form of a drum cylinder, rotates relative to casing 12, and puncturing member 26. In addition, puncturing member 26, rotates incrementally relative to reservoir member 18. Puncturing member 26 contains a plurality of cutting and/or piercing instruments 34 spaced apart from one another and disposed radially about an outer surface of puncturing member 26. Rotation of puncturing member 26 relative to reservoir member 18 causes sequential cutting and/or piercing of a seal for a particular aptamer-containing cavity 20. A gear mechanism 40 and 42 located at one end of puncturing member 26 and at the corresponding end of reservoir member 18 causes puncturing member 26 and reservoir member 18 to rotate relative to one another so as to permit the cavities to be opened in a timed sequence. Once a seal is cut and/or pierced, the aptamer and/or aptamer-microsphere formulation exits the cavity and passes out of casing 12 via aperture 14. The puncturing member 26 and reservoir member 18 then rotate relative to one another so that a different cutting and/or piercing instrument 34 is brought into contact with different seal of a different cavity 20. The speed of rotation of puncturing member 26 and reservoir member 18 relative to one another can be adjusted to cause the release of the microspheres over a desired period of time.

In one embodiment, drive mechanism 24, comprises a U-shaped pivotable member pivotably coupled to reservoir member 18. During operation, the pivotable member pivots about the drum, the motion of which is coupled, for example, via a ratchet and path mechanism, to reservoir member 18 so as to positively drive reservoir member 18 in unilateral increments about its axis of rotation. The incremental rotation of reservoir member 18, in turn, positively drives rotation of puncturing member 26 via, for example, interfitting gear components 40 and 42.

U-shaped pivotable member preferably comprises one or more permanent magnets disposed within the U-shaped portion of fee pivotable member (for example, two permanent magnets facing one another and each disposed on each side of the U-shape). Motion can be induced by induction of a magnetic field in the vicinity of the permanent magnets, thereby inducing their motion in one way or another. The magnetic field can be created by periodically passing current through an immobilized coil. For example, the immobilized coil may be attached to the interior of casing 12, and positioned so that at certain times, for example, when no magnetic field is generated by the coil, the U-shaped pivotable member can return to a position in which the coil is disposed within the central void defined by each arm of the. U-shaped member.

In an exemplary transcleral drug delivery device, the device comprises a casing, which preferably has an eye contacting surface (i) complementary in shape to the outer surface of the eye and (ii) defines an aperture port running therethrough. As a result, the aptamer and/or the aptamer containing microspheres exit the casing via the aperture port and contact the enter surface of the eye in the vicinity of the aperture port. The drug delivery device can be attached to the eye using routine surgical or medical procedures. For example, the device may be attached to the outer surface of the eye via, for example, tissue adhesive, scleral flaps, suture techniques, or a combination thereof.

When tissue adhesive is used, the adhesive is applied to the eye contacting surface of the casing, the contact surface of the eye, or both, and then the device is attached to the outer surface of the eye. A preferred tissue adhesive includes feobutyl cyanoacrylate adhesive available from Braun, Melsunger, Germany, and Ellman International, Hewlett, N.Y. in addition, tissue adhesive may be used to seal the edge of the device casing to the solera. Also, the tissue adhesive may be used to secure scleral flaps to outer portions of the device easing.

In the scleral flap approach, partial thickness scleral flaps are created using a surgical blade, such as, a 57 Beaver blade. The flaps preferably are of a width to cover at least a portion of the outer casing of the device, in an embodiment, the tissue contacting surface of the device casing may optionally contain a rim or flange extending around the casing so that the scleral flap can be wrapped over and then attached to the rim or flange. Once the device is positioned, the scleral flaps can be sutured to each other and/or glued to the device easing using tissue adhesive.

In the suturing approach, sutures are passed through partial thickness sclera and then through correspondingly located aperture holes, eyelets or rings disposed m the device casing. Sutures preferably are preplaced if adhesive is to be used in conjunction with suturing. Sutures useful for immobilizing the device include, for example, 4-0 or 5-0 monofilament nylon, silk, mersilene or polyester. Once the device is positioned, the sutures then are permanently secured.

Furthermore, if desirable the portion of the sclera that contacts the device casing, and more preferably the portion of the sclera located adjacent to the aperture port of the casing, may he thinned prior to attachment of the device. Thinning may be accomplished using a surgical blade or a laser, for example, an Erbium YAG laser.

The desired rate of aptamer delivery will depend upon the age, sex, and weight of the recipient, as well as the particular aptamer and the disorder to be treated. The choice of a particular aptamer, the rate and mode of administration, and site of implantation are within the level of skill in the art. for example, aptamer may be administered at doses ranging, for example, from about 0.001 to about 500 mg/kg, more preferably from about 0.01 to about 250 mg/kg, and most preferably from about 0.1 to about 100 mg/kg. Using such a device, aptamer may be administered periodically as boluses. Thereafter, the microspheres break down over time to release the aptamer over a prolonged period of time to simulative continuous not bolus administration.

To the extent that the aptamer containing device becomes exhausted, for example, runs out of power and/or aptamer, the device may he removed. A new device may then be attached to the site of interest or the old device, once fabricated with a new power source and/or new aptamer containing microspheres, may be reimplanted at the site of interest.

The present invention may be further understood by reference to the following non-limiting examples.

### Example 1

### Controlled Delivery of an Anti-VEGF Aptamer with Poly(lactic-co-glycolic) Acid Microspheres

The following example demonstrates the effectiveness of a drug delivery modality that releases an anti-VEGF aptamer, EYE001, in a sustained and controlled manner over a significant period when applied locally to the outer part of the sclera. The retina and choroid are the target tissues, because this aptamer, as discussed above, blocks the contribution of VEGF to choroidal neovascularization and diabetic macular edema. Use of transscleral administration, no more frequently than every 6 weeks, is an attractive substitute to intravitreal injections of the naked, unencapsulated aptamer.

As is discussed below, PLGA microspheres containing anti-VEGF RNA aptamer (EYE001) formulations in the solid-state were developed by an oil-in-oil solvent evaporation process. In vitro experiments were performed to characterize the release profiles of this formulation. Stability and bioactivity of the released drug was assayed by monitoring the aptamer's ability to inhibit VEGF-induced cell proliferation in human umbilical vein endothelial cells (HUVECs). Cell proliferation, experiments were conducted with aptamer aliquots collected after short-, mid-, and long-term release time points. To demonstrate the feasibility of this polymer device as a potential transscleral delivery device, an in vitro apparatus was developed to assess polymer hydration and degradation through rabbit sclera and subsequent delivery through it. The results of these studies showed that PLGA microspheres are able to deliver EYE001 in a sustained manner at an average rate of 2 μg/day over a period of 20 days. Solid-state stabilization of the aptamer with the disaccharide trehalose before lyophilization and encapsulation in PLGA rendered the drug more stable after release. Cell proliferation experiments demonstrated that the bioactivity of the aptamer was preserved after release, as indicated by inhibition of endothelial cell proliferation after incubation with VEGF. Microspheres packed into a sealed chamber and placed onto the “orbital” part of a rabbit sclera for a period of 6 days became hydrated and started to degrade, as shown by scanning electron microscopy (SEM). As a result, the aptamer was delivered from the microspheres through the sclera, as determined spectrophotometrically. These experiments demonstrated that it is possible to load aptamer-containing microspheres into a device and that the resulting device can be placed on the orbital surface of the sclera.

The following data also demonstrate the feasibility of delivering the anti-VEGF aptamer EYE001 in a sustained and controlled manner and in a biologically active form.

A. Production of Lyophilized RNA Aptamer EYE001

EYE001 was developed by Gilead Sciences, Inc. (Boulder, Colo.) by the systematic evolution of ligands by exponential enrichment (SELEX) process as described (Ruckman et al. (1998) J. BIOL. CHEM. 223:20556-20567; Costantino et al. (1998) J. PHARM. SCI. 87:1412-1420). EYE001 is a pegylated RNA aptamer of 50 kDa, with an A-type secondary structure, 40 mg/mL solubility, and a net negative charge of −28. The structure of EYE001 is as follows:

5′-[40 kd PEG]-[HN-(CH₂)₅O]-pC_{f}pGₘpGₘpAᵣpAᵣpU_{f}pC_{f}pAₘpGₘ
pU_{f}pGₘpAₘpAₘpU_{f}pGₘpc_{f}pU_{f}pU_{f}pAₘpU_{f}pAₘpC_{f}pAₘpU_{f}pC_{f}pC_{f}pGₘ
3′-p-3′dT.

The 40 kd PEG component represents two 20 kilodalton-poly(ethylene glycol) polymer chains covalently attached to the two amine groups on a lysine residue via carbamate linkages. This moiety is in turn linked to the oligonucleotide via the amino linker, [HN—(CH₂)₅O—], a bifunctional amino linker. The linker is attached to the oligonucleotide by a standard phosphodiester bond; p represents the phosphodiester functional groups that link sequential nucleosides and that link the amino linker to the oligonucleotide. All of the phosphodiester groups are negatively charged at neutral pH and have a sodium atom as the counter ion; Gₘ or Aₘ and C_{f} or U_{f} and Aᵣ represent 2′-methoxy, 2′-fluoro and 2′-hydroxy variations of their respective purines and pyrimidines; C, A, U, and G is the single letter code for cytidylic, adenylic, uridylic, and guanylic acids. All phosphodiester linkages of this compound, with the exception of the 3′-terminus, connect the 5′ and 3′ oxygens of the ribose ring. As shown, the phosphodiester linkage between the 3′-terminal dT and the penultimate Gₘ links their respective 3′-oxygens. This is referred to as a 3′, 3′ cap.

Samples were lyophilized (in model SNL315SV; Savant Instruments, Farmingdale, N.Y.) at a chamber pressure of 80 mbar and & shelf temperature of −45° C. for 48 hours to obtain excipient-free aptamer. The lyophilized material then was sealed in sterilized glass vials and stored at −20° C. until use. Lyophilized samples containing trehalose (Sigma Chemical Co., St. Louis, Mo.) at a 1:3 weight ratio were prepared by adding an appropriate amount of concentrated excipient solution to the excipient-free aptamer solution before lyophilization (Costantino et al. (1998) J. PHARM. SCI. 87:1412-1420; Carrasquillo et al. (2000) BIOTECH. APPL. BIOCHEM. 31:41-53.) The ratio was selected based on the mass amounts of aptamer to trehalose needed to stabilize aptamer structure and function on lyophilization and thus prevent lyophilization-induced structural changes.

B. Microsphere Preparation

PLGA microspheres were prepared by a non-aqueous oil-in-oil method (see, Carrasquillo et al. (2001) J. CONTROL RELEASE 26:109-208). Briefly, 25 to 30 mg of solid aptamer was suspended in a solution of 200 mg/2 mL PLGA (Resomer 502 H, i.v. (inherent viscosity) 0.16-0.24 dL/g, 0.1% in chloroform, 25° C., molecular weight [Mw] 10 to 12 kDa, half-life for degradation approximately 1 to 15 months; Boehringer Ingelheim Pharma KG, Ingelheim, Germany) in methylene chloride with a homogenizer (Polytron, model PT 1200C; Brinkman, Westbury, N.Y.) using a standard 12-mm diameter generator at approximately 20,000 rpm for 1 minute. After suspension of the aptamer, the coacervating agent poly(dimethylsiloxane) was added at a rate of 2 mL/min under constant homogenization, to ensure homogeneous dispersion of the coacervating agent, phase separation of PLGA dissolved in methylene chloride, and formation of microspheres. The coacervating mixture containing the microspheres then was poured into an Erlenmeyer flask containing 50 mL heptane under constant agitation and stirred for 3 hours at room temperature to allow for hardening of the microspheres. Microspheres were collected by filtration with the use of a 0.22-μm nylon filter, washed twice with heptane, and dried for 24 hours at a vacuum of 80 mbar.

C. Encapsulation Efficiency

Encapsulation efficiency was determined as described (Carrasquillo et al. (2001) J. PHARM. PHARMACOL. 53:115-120). Briefly, ten milligrams of PLGA microspheres was placed in 2 mL methylene chloride and stirred for 30 minutes to dissolve the polymer. The solution then was centrifuged at 10,000 rpm for 10 minutes to precipitate the insoluble RNA aptamer. The resulting supernatant was removed, and the remaining methylene chloride allowed to evaporate. To ensure evaporation of the methylene chloride, the sample was placed in a vacuum for 24 hours. The aptamer then was dissolved in Dulbeceo's phosphate-buffered saline (DPBS; GibcoBRL, Grand Island, N.Y.), and the concentration of entrapped aptamer in PLGA determined spectrophotometrically. The percentage encapsulation efficiency was calculated by relating the experimental aptamer entrapment to the theoretical aptamer entrapment: (experimental/theoretical)×100.

D. Characteristics of PLGA Microspheres

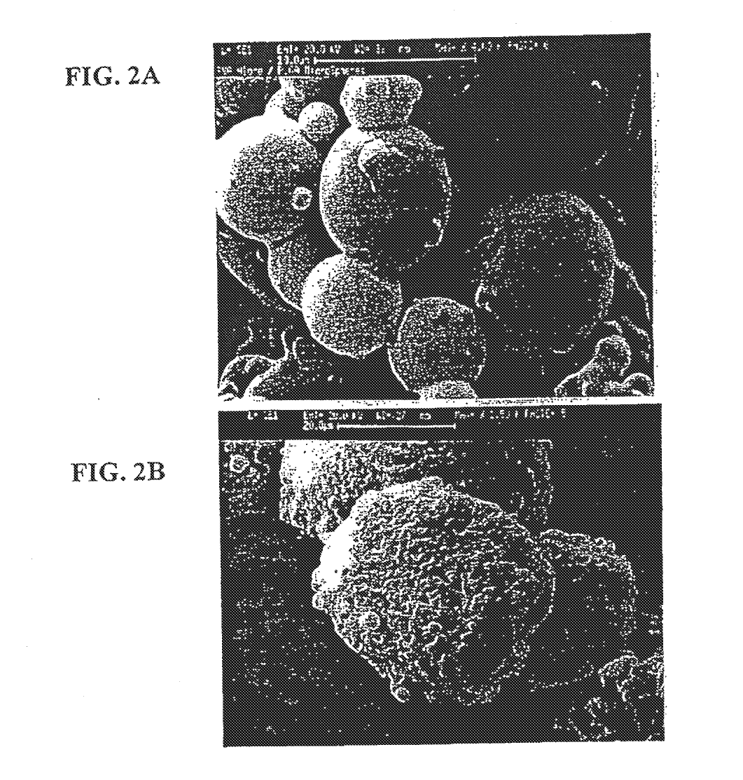

Images of the microspheres, obtained by scanning electron microscopy (SEM), after preparation indicated the formation of nonporous spheres with an average diameter of 14±4 (see. FIG. 2A) and 16±4 μm (see, FIG. 2B) after hydration. SEM images were obtained as Follows. Samples were affixed with double-sided carbon tape to an aluminum stub and sputtered with approximately 100 nm gold (Sputter Coating System; SPI, West Chester, Pa.). SEM images were then obtained (model S360; Cambridge Instruments, Mousey, N.J.). The encapsulation efficiencies of aptamer into PLGA varied with the original amount of drug used as starting material. The encapsulation efficiency for microspheres containing aptamer colyophilized with trehalose was 80% ±5% when 32.1 mg was used as the starting material, whereas for excipient-free aptamer-containing microspheres, the encapsulation efficiency was 71% ±2% when 6.6 mg was used. Analysis of the microspheres after 10 days of release showed degradation of the polymer matrix and the formation of pores through which the aptamer was slowly released (see, FIG. 2B).

E. RNA Aptamer EYE001 Release from PLGA Microspheres

In vitro release profiles were studied as follows. Ten milligrams of solid microspheres was placed in 2 mL of DFBS, 1×(pH 7.3) and incubated at 37° C. Every 24 hours, the microspheres were centrifuged gently at 500 rpm for 1 minute, and the supernatant was removed for determination of aptamer concentration at 260 nm, ε_{PBS}=25.08 cm⁻¹) (mg/mL)⁻¹ as described (Carrasquillo et al. (2001) J. CONTROL RELEASE 76:199-208; Jones et al. (1995) J. MED. CHEM. 38:2138-2144). Microspheres then were resuspended in 2 mL, fresh DPBS to maintain sink conditions and to control the pH (Carrasquillo et al. (2001) J CONTROL RELEASE 26:199-208; Park et al. (1995) J. CONTROL RELEASE 33:211-222). Ten milligrams of blank (empty) PLGA microspheres were subjected to the same conditions as PLGA-loaded microspheres, and the supernatant collected from these was used as a blank in the spectrophotometric analysis. Data are presented as the average of three independent experiments with standard deviations.

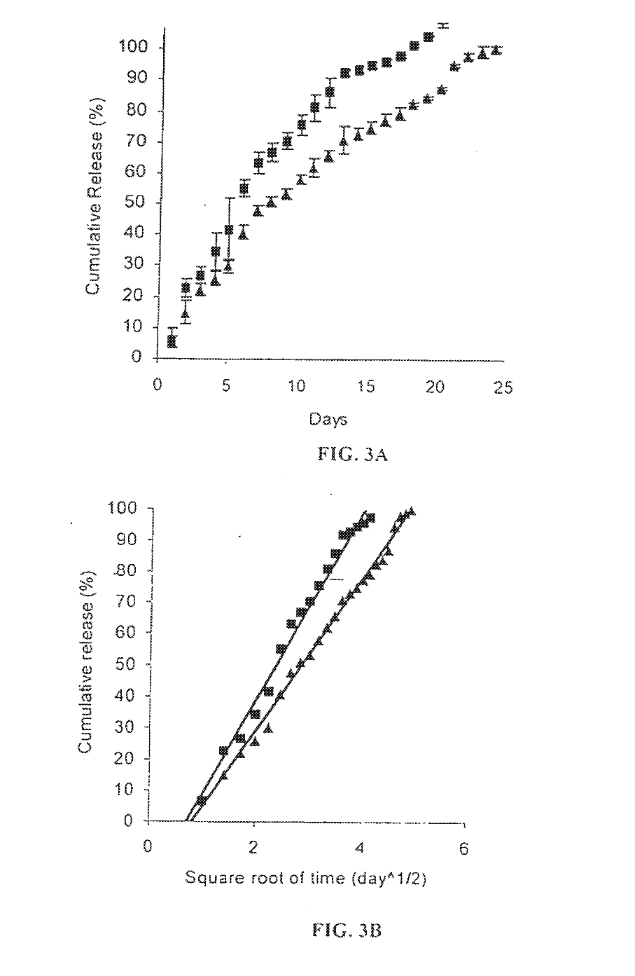

In vitro release profiles (see. FIG. 3A) for both excipient-free aptamer and aptamer colyophilized with trehalose at a 1:3 weight ratio of aptamer to trehalose (herein referred to as EYE001-Tre) exhibited a controlled release of the drug in a period of more than 20 days. Release kinetics were characterized by a very low-burst release during the first 24-hour period, followed by a continuous release with no evidence of a lag phase. Both formulations were completely released, indicating no adsorption of the aptamer to the polymer core. The average amount of drug released was 2 μg/day, regardless of the amount of drug originally encapsulated. The encapsulation efficiency for both formulations in PLGA was 70% to 85%, with a theoretical loading of 3.95% and actual loading of 2.76% ±0.80%, indicating that the presence of trehalose had no effect in the encapsulation efficiency of the polymeric system.

As discussed, the release profiles of EYE001 from these microspheres were characterized by a low initial burst, followed by continuous release in the absence of a lag phase. Typical release profiles from PLGA microspheres are triphasic, characterized by an initial burst as drug entrapped near the surface releases, followed by a lag phase controlled by polymer degradation and final release of the drug as it diffuses from the polymer core as erosion takes place (Batycky et al. (1997) J. PHARM. SCI. 86:1464-1477). In the scenario observed, it appears that EYE001 formulations encapsulated in PLGA were homogeneously distributed throughout the polymeric matrix. The process is described by the following equation:

Q=√2WDCₛt

where Q is the rate of released drug, D is the diffusion coefficient of the drug in the matrix, W is the total amount of the drug per unit volume of matrix, Cₛ is the solubility of the drug in the matrix, and t is the drug release time.

The release of both excipient-free aptamer and EYE001-Tre from PLGA as a function of the square root of time (t_{1/2}) showed a linear relationship with correlation coefficients of 0.98 and 0.99 respectively (see, FIG. 3B). These data support the hypothesis that both aptamer formulations were released through a diffusion-controlled process. An important consideration in the development of a long term delivery device for a nucleic acid such as EYE001 is its stability before, during, and after the encapsulation process in PLGA. Nucleic acids are known to suffer deportation and become susceptible to free radical oxidation in aqueous solutions (Lindahl (1993) NATURE 362;709-715; Demple et al. (1994) ANNU REV BIOCHEM. 63:915-948). In an attempt to obviate this, EYE001 was colyophilized with the stabilizer trehalose via a completely nonaqueous oil-in-oil method (Carrasquillo et al. (1998) PHARM. PHARMACOL. COMMUN. 4:563-571; Schwendeman et al. (1996) STABILITY OF PROTEINS AND TTHEIR DELIVERY FROM BIODEGRADABLE POLYMER MICROSPHERES, New York: Marcel Dekker) for the creation of polymer microspheres that has been effective in the delivery of biologically active proteins with native secondary structures (Carrasquillo et al. (2001) J. CONTROL RELEASE 76:199-208; Carrasquillo et al. (1998) PHARM. PHARMACOL. COMMUN. 4:563-571; Schwendeman et al. (1996) STABILITY OF PROTEINS AND THEIR DELIVERY FROM BIODEGRADABLE POLYMER MICROSPHERES, New York Marcel Dekker; Ando et al. (1999) J. PHARM. SCI. 88:126-130; Sanchez et al. (1999) INT. J. PHARM. 185:255-266.

F. Secondary Structural Determination of EYE001 Formulations upon Lyophilization

To assess any structural changes due to the nature of the formulation of EYE001 upon Lyophilization EYE001 formulations lyophilized as described hereinabove were reconstituted in PBS and its circular dichroism (CD) spectra determined and compared with an aqueous EYE001 standard. CD spectra were recorded on a CD spectrometer.(model 202; Aviv Instruments, Lakewood, N.J.). Data were collected at 25° C using a bandwidth of 0.5 nm and an average time of 0.1 second. The CD spectra, were collected from 200 to 330 nm with a 0.5 cm quartz cells and corrected fox the phosphate buffer signal contribution measured under identical conditions.

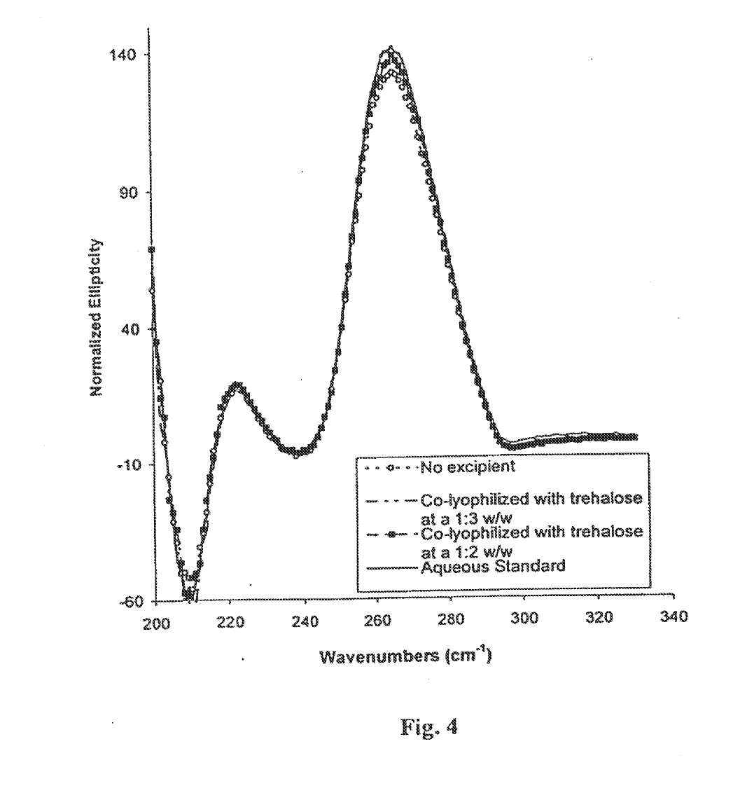

Given that EYE001 has as A-type RNA structure (duplex formation, right-banded helix) (Ruckman et al. (1998) J. BIOL. CHEM. 273:20556-20567), the CD spectra exhibit a maximum of approximately 260 nm and a minimum of approximately 210 nm (Shelton et al. (1099) BIOCHEM. 38:16831-16839; Carmona et al. (1999) BIOCHIM. BIOPHYS. ACTA 1432:222-233). A decrease in molar ellipticity in either maxima or minima is a reflection of a secondary structural change (Shelton et al. (1999) BIOCHEM. 38:16831-16839; Carmona et al. (1999) BIOCHIM. BIOPHYS. Acta 1432:222-233). The CD spectrum of EYE001 in the absence of any excipient on lyophilization and further reconstitution exhibited a slight decrease in intensity at both wavelengths. It was observed that when increasing the mass ratio of the disaccharide stabilizer trehalose (Carrasquillo et al. (1999) J. PHARM. SCI. 88:166-173; Carrasquillo et al. (2001) J. PHARM. PHARMACOL. 53:115-120; Carrasquillo et al. (2001) J. CONTROL RELEASE 76:199-208; Costantino et al. (1998) J. PHARM. SCI. 87:1412-1420; Carrasquillo et al. (2000) BIOTECH. APPL. BIOCHEM. 31:41-53) to EYE001 before lyophilization, there was an improvement in the retention of structure, as evidenced by molar ellipticities at both wavelengths comparable with those of the aqueous EYE001 standard (see, FIG. 4).

G. Anti-VEGF Aptamer Activity after Release from PLGA

EYE001 activity after encapsulation and former release from PLGA microspheres was assayed by monitoring its ability to inhibit VEGF-induced proliferation of human umbilical vein endothelial cells (HUVECs). HUVECs were obtained from Cascade Biologics, Inc. (Portland, Oreg.), and were maintained in growth-factor supplemented medium, including 2% vol/vol fetal bovine serum (FBS), 1 μg/mL hydrocortisone, 10 ng/mL human epidermal growth factor, 3 ng/mL basic fibroblast growth factor, and 10 ng/mL heparin under standard tissue culture conditions (5% CO₂, 37° C., 100% relative humidity). Medium was changed every 48 to 72 hours, and cells were passaged by standard trypsinization and plated at a cell concentration of 2.5 ×10³ cells/cm².

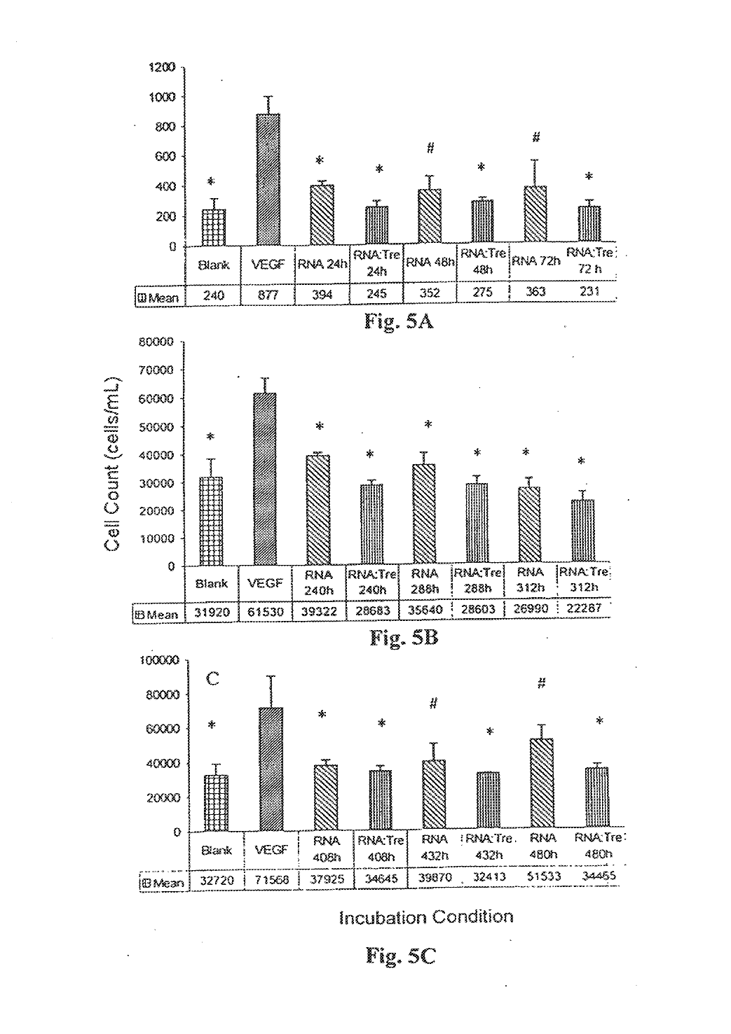

To determine the feasibility of polymer microspheres as a viable delivery device, proliferation assays were conducted at various stages during the release period. The representative time points chosen were at early (24-72 hours as shown in FIG. 5A), intermediate (240-312 hours as shown in FIG. 5B), and late stages (408-480 hours as shown in FIG. 5C) of release. Proliferation assays were performed as described (Ruckman et al. (1998) J. BIOL. CHEM. 273:20556-20367) with the following modifications. HUVECs were seeded into 6-well or 12-well plates (2.5×10³ cells/cm²) as required in growth-factor-deficient medium (Medium 200; 5% FBS, 1 μg/mL heparin; Cascade Biologics) for 24 hour before experimentation. Aptamers 10 nM) were collected after release from PLGA microspheres at specific time points and then VEGF₁₆₅ (10 ng/mL; R&D Systems, Minneapolis, Minn.) was added to cells and incubated for 4 days. Cells were trypsinized and counted with a cell counter (model Z1; Coulter, Beds, UK). Wells containing cells without addition of aptamer or VEGF₁₆₅ were trypsinized and counted for basal growth estimation (represented in FIGS. 5A-5C as “Blank”). The paired Student's two-tailed t-test was used to compare cell counts after each incubation condition. An α level of 0.05 was used as the criterion to reject the null hypothesis of equality of means.

The resulting data is summarized in FIG. 5, wherein samples pretreated with VEGF and then incubated with aptamer released from microspheres lacking or containing trehalose are denoted as “RNA” or RNA-Tre, respectively. The number next to or beneath each designation represents the length of time the aptamer was released from the microsphere. The VEGF-induced proliferation of HUVECs after a period of 4 days (represented in FIGS. 5A-5C as “VEGF”) showed a three-fold average increase in cell counts compared with those found in the Blanks,

On HUVEC incubation with VEGF in the presence of the different aptamer formulations after release, it was evident that regardless of the formulation state, the aptamer was capable of at least partially inhibiting VEGF-induced ceil proliferation (see, FIG. 5). It is worth noting that the inhibition showed by the aptamer was, in general, enhanced when EYE001 was colyophilized in the presence of trehalose and then encapsulated in PLGA. EYE001 preserved its bioactivity after encapsulation in PLGA and during its release over a period of 20 days (see, FIGS. 5A-5C). Incubation of HUVECs with PLGA-loaded microspheres containing EYE001 formulations and degraded PLGA supernatant had no effect on HUVEC proliferation (data not shown).

Incubation of PLGA microspheres directly with HUVECs revealed the same trend as that of the aptamer collected after it was released from isolated microspheres in vitro. No evident signs of toxicity or cell death were observed when blank PLGA microspheres were incubated with HUVECs from microscopic observations and cell counts (data not shown). These results are consistent with reports by others who have conducted cell proliferation assays with polylactides of various molecular weights with rat epithelial cells, human fibroblasts, and oteosacroma cells under culture conditions (van Sliedregt et al. (1992) J. MATER. SCI. MATER. MED. 3:365-370). Overall, it was determined that satisfactory biocompatibility was exhibited (van Sliedregt et al. (1992) J. MATER. SCI. MATER. MED. 3:365-370; Athanasiou et al. (1996) BIOMATERIALS 17:93-102). These data support the conculsion that the method described in this Example is useful for the long-term inhibition of VEGF-mediated responses in vivo.

H. Transscleral Delivery of EYE001 Released from PLGA Microspheres

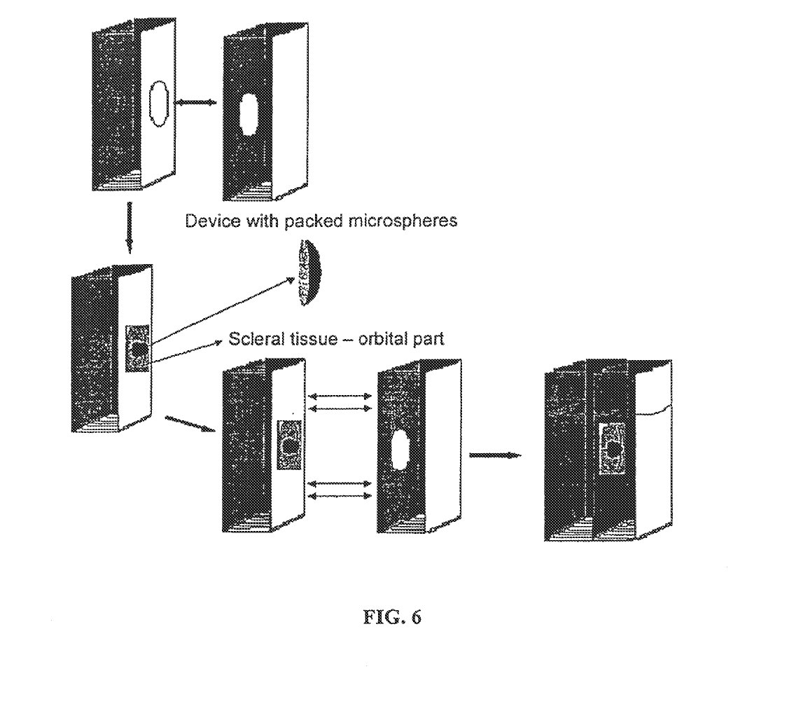

Active EYE001 was delivered front PLGA microspheres in a controlled manner for an extended period in vitro. It is also of interest whether the hydration of the sclera would be sufficient to degrade the microspheres and result in aptamer release and diffusion through the sclera. PLGA-loaded microspheres were loaded into a device, which was then placed on the sclera of Dutch belted rabbits as shown in FIG. 6.

The following experiments were performed pursuant to the ARVO Statement for the Use of Animals in Ophthalmic and Vision Research and guidelines developed by the Animal Care Committee of the Massachusetts Eye and Ear Infirmary. Dutch belted rabbits (Myrtle's Rabbitry, Inc., Thompson Station, Tenn.), each weighing 2 to 3 kg were anesthetized and killed with an intramuscular combination of 40 mg/kg ketamine (Abbott Laboratories, North Chicago, Ill.) and 10 mg/kg xylazine (Bayer, Shawnee Mission, Kans.) as described (Ambati et al. (2000) INVEST. OPHTHALMOL. VIS. SCI. 41:1181-1185). The eyes were enucleated immediately before the rabbits were killed and were immersed in DPBS. The adherent muscles were excised, and scleral tissue was removed. Areas free of nerve and vessel emissaries were used to obtain 12×20-mm slices of sclera under microscope caliper guidance. Each piece of sclera was immersed on PBS and used on the day of isolation.

The in vitro apparatus used for these experiments was modified from one previously described (see, FIG. 6) (Ambati et al. (2000) INVEST. OPHTHALMOL. VIS. SCI. 41:1181-1185). Briefly, a 10×18 -mm window was created on one face of a polystyrene cuvette (Sigma, St. Louis, Mo.) with use of a vertical milling machine (Bridgeport Machines, Bridgeport, Conn.), and a piece of sclera was blotted dry and placed over this window without stretching, avoiding asymmetrical stress. The tissue was sealed to the cuvette with: a small amount of cyanoacrylate tissue adhesive (Ellman International, Hewlett N.Y.) applied continuously around its rim..

PLGA-loaded solid microspheres (5 mg) were packed into a device 9 mm in diameter and 4 mm in depth made from a polypropylene cap of a 26.5-gauge needle (BD Biosciences, Lincoln Park, N.J.). Cyanoacrylate tissue adhesive was placed around the border of the device and scaled against the orbital surface. A second identical cuvette was aligned wish the first cuvette and glued in place along the margins of the tissue. Both sides of the cuvette then were filled with DPBS (2.5 mL), and the apparatus was placed in an incubator at 37° C. without agitation. One side was considered the “uveal” chamber where diffusions of the aptamer would occur if the delivery were successful. The other side facing the “orbital” surface of the sclera would comprise any part of the sclera not covered by the device containing the microspheres and would serve as a control to assess any leakage from the device. A protease inhibitor cocktail (Complete Mini; Roche Diagnostics, Indianapolis, Ind.), at concentrations recommended by the manufacturer, was added so avoid proteolytic degradation of the tissue. In addition, 0.1 mM sodium azide was added to inhibit growth of bacteria in the medium (Boubriak et al. (2000) EXP. EYE. RES. 71:503-514). After 24 hours, the “uveal” chamber was sampled for aptamer concentration at 260 nm with a spectrophotometer (UV-Vis LambdaBio 40; Perkin Elmer, Wellesley, Mass.), and the “orbital” chamber was sampled as a control. Each side was replenished with fresh DPBS. To assess microsphere hydration and degradation, scleral tissue was analyzed by SEM after incubation for a determined period.

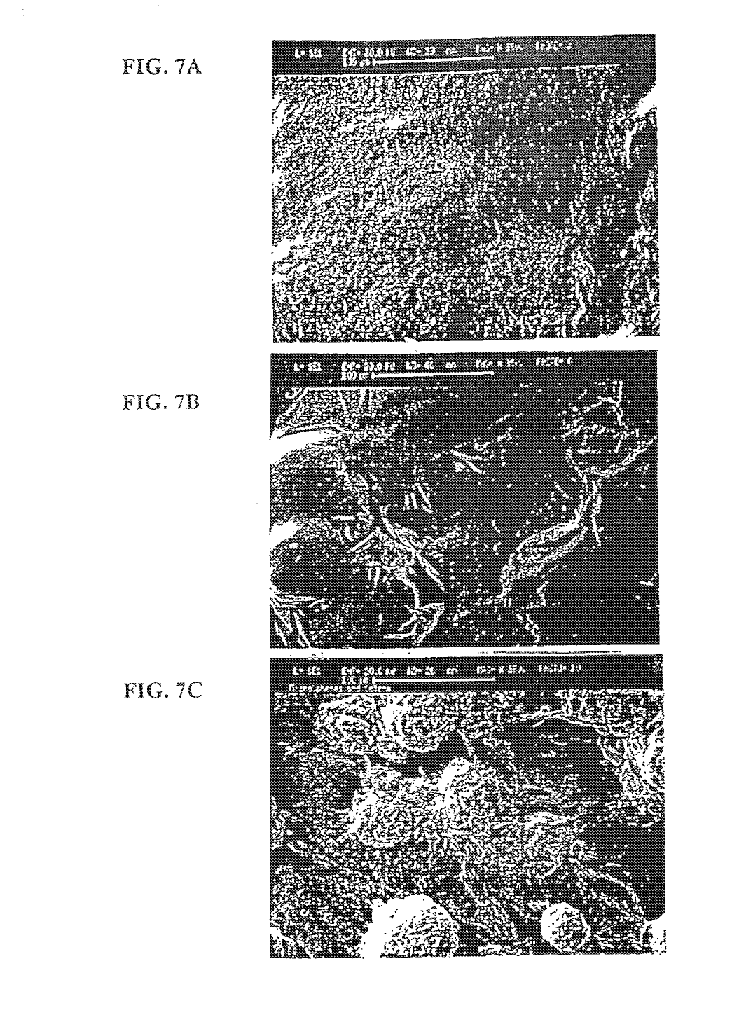

The degree of polymer degradation was monitored qualitatively by analyzing the morphology of the microspheres. SEM pictures showed the morphologic state of the microparticles after exposure to scleral hydration alter a period of 18 hours and after 6 days. FIG. 7A is an SEM of scleral tissue prior to the addition of microspheres. During the first 18 hours, the polymer microspheres seemed to adhere to the tissue,, but no significant degradation was observed (see, FIG. 7B), as expected, because of the short incubation time. However, after 6 days, PLGA microspheres showed significant degradation and formation of pores along its surface (see, FIG. 7C). The visible signs of degradation indicated that scleral hydration was sufficient to degrade the PLGA-loaded microspheres, indicating feasibility of the delivery method for EYE001 through the sclera.

To determine whether diffusion of EYE001 through the sclera was indeed possible after delivery from PLGA microspheres, the aptamer concentration was monitored in the uveal chamber (sampling the chamber with the uveal side of the sclera exposed), and, as a control the aptamer concentration in the orbital chamber was monitored (sampling chamber with the orbital side of sclera exposed and containing the device loaded with microspheres). Having determined the characteristics of the in vitro release profiles of EYE001 from the microspheres, aptamer diffusion through the sclera was monitored for 6 days. Table 1 presents the data showing the amount of aptamer diffused through the sclera. The amount of aptamer delivered from PLGA microspheres and diffused through the sclera is comparable with that released in vitro from isolated microspheres. An average of 2 μg/day was sampled in the uveal chamber, indicating that EYE001 diffused readily through the sclera, as reported previously for molecules of similar molecular weight. An average of 0.5 μg/day was sampled in the control chamber. SEM analysis of lyophilized powder obtained after freeze drying of the volume sampled in the uveal chamber revealed that there were no microspheres present, indicating that the drug permeated in its free, nonencapsulated form.

TABLE 1

| | EYE001_{SC}* | EYE001_{CC}^{†} |
|---|---|---|
| Day | (μg) | (μg) |
| 1 | 3.4 ± 0.8 | 0.7 ± 0.3 |
| 2 | 2.3 ± 0.5 | 0.5 ± 0.3 |
| 3 | 2.1 ± 0.6 | 0.5 ± 0.2 |
| 4 | 1.8 ± 0.3 | 0.6 ± 0.4 |
| 5 | 2.4 ± 0.1 | 0.5 ± 0.2 |
| 6 | 2.6 ± 0.2 | 0.4 ± 0.3 |

Data are presented as the average results of three experiments ±SD.
*Sampling Chamber.
^{†}Control Chamber.

Given that diffusion was monitored for 6 days in an in vitro setup, an important consideration was the integrity and viability of the sclera during the transport study. To examine this, cultured scleral tissue immersed in PBS and incubated at 37° C. for 6 days was examined by transmission electron microscopy (TEM). Tissue was placed in modified Karnovsky fixative consisting of 2.5% glutaraldehyde and 2% formaldehyde in 0.1 M cacodylate buffer with 8 mM CaCl₂ and fixed for 12 to 24 hours at 4° C. The specimens subsequently were changed to 0.1 M cacodylate buffer for storage at 4° C. The tissue then was trimmed to block size and postfixed in 2% aqueous OsO₄ for 2 hours at room temperature. After the tissue was rinsed in buffer, it was dehydrated in ascending concentrations of ethanol, transitioned through propylene oxide, and infiltrated with mixtures of propylene oxide and Epon (EMBed 812; Electron Microscopy Sciences, Fort Washington, Pa.), embedded in pure Epon, and polymerized at 60° C. for 18 to 24 hours. One-micrometer sections and thin sections were cut on an ultramicrotome (Ultracut E; Leica, Deerfield, Ill.). The 1 μm sections were stained with 0.5% toluidine blue and the thin sections with saturated aqueous many uranyl acetate and Sato lead stain, and then examined with a transmission electron microscope (model CM-10 Philips, Eindhoven, The Netherlands).

As a control fresh scleral tissue, fixed the same day it was detached, was analyzed. There were signs of swelling of the collagen fibrils in the cultured sclera when compared with fresh rabbit sclera, as evidenced by the thickness of the collagen fibers, hut the general ultrastructure of the tissue was preserved, as determined by TEM. This is consistent with the observations in other investigations in which similar in vitro experiments were performed to determine diffusion of solutes through the sclera, with the results indicating that normal scleral physiology can be maintained over the course of short- and long-term perfusion periods (Geroski et al. (2000) INVEST. OPHTHALMOL. VIS SCI. 41:961-964).

Rabbit sclera is 71% water (Boubriak et al. (2000) EXP. EYE RES. 71:503-514) and, as documented by electron microscopy (see, FIG. 7C), it served to hydrate and degrade the solid PLGA microspheres placed on the orbital side of the sclera, which were not in contact with any hydration medium other than the hydrated scleral surface itself. An important aspect of PLGA controlled-delivery devices is that they provide continuous release and avoid the repeated use of injections or high concentrations of drug to achieve the desired pharmacological response. Even though controversy exists over how the flux over the sclera occurs and whether it achieves steady state (Prausnitz et al. (1998) IND. ENG. CHEM. RES. 37:2903-2907), the controlled-drug delivery device discussed hereinabove increases drug-sclera contact, thus improving scleral absorption. The hypocellularity (Foster et al. (1994) THE SCLERA, New York: Springer-Verlag) and large surface area (Olsen et al. (1998) AM J OPHTHALMOL. 125:237-241) of the human sclera, as well as its remarkable tolerance of foreign bodies overlying its surface (e.g., scleral buckles) helps to facilitate diffusion through it and allow a long-term transscleral delivery device to be clinically feasible (Haynue et al. (1994) PRINCIPLES AND PRACTICE OF OPHTHALMOLOGY Philadelphia: WB Saunders).

### Example 2

### Anti-VEGF Aptamer Reduces Blood Vessel Leakage In Vivo

This example shows that the EYE001 aptamer, when released from a microsphere, can traverse the sclera and then impart a biological effect within the eye.

EYE001 aptamer was encapsulated within poly(laetic-co-glycolic) acid (PLGA) microspheres using an oil-in-oil solvent evaporation process. Briefly, 25-30 mg of lyophilized EYE001 was suspended by homogenization in a 2 mL solution of PLGA (200 mg) dissolved in methylene chloride. Two mL of the coacervating agent poly(dimethylsiloxane) was added to the suspension at a rate of 2 mL/min and homogenized for 1 minute at 2,000 rpm. The resulting oil-in-oil suspension was added to 50 mL of heptane under constant agitation and stirred for 3 hours to allow microsphere hardening and methylene chloride evaporation. Microspheres were collected by filtration and lyophilized for 24-48 hours for further methylene chloride evaporation. Prepared microspheres were subsequently stored at −20° C. until use.

Prior to delivery, EYE-001 aptamer containing microspheres were packed into a polypropylene chamber. Cyanoacrylate glue was placed on the border of the chamber and the chamber was adhered onto the left eyes (OS) of dutch-belted rabbits at a location about 5 mm away from the limbus of each eye. the PLGA present in the packed microspheres, when in fluid communication with the highly hydrated sclera, degraded to release the nucleic acid aptamer from the microspheres. The devices attached to each left eye were left in place for one or two weeks. The right eye of each rabbit (OD) was used as a control (i.e., no EYE-001 aptamer).

The day before analysis, each eye received an intravitreal injection of 1 mg/mL of VEGF (R&D Systems) to trigger vascular permeability of the blood vessels within the eye. On the day of analysis, the rabbit femoral vein was cannulated with a 24 gauge catheter and Evans Blue dye was infused into the bloodstream over 10 seconds at a dosage of 45 mg/kg. 2 hours after infusion of Evans Blue dye, 1 mL of blood was drawn from the left ventricle to obtain a final concentration of Evans Blue dye in circulation. After 4 hours circulation time, the chest cavity was opened and the animals were perfused through the left ventricle at 37° C. with 400 mL of citrate buffer (0.05M, pH 3.5) and subsequently with 500 mL of citrate-buffered paraformaldehyde (1%-wt/vol, pH 3.5, Sigma). Immediately after perfusion (physiological pressure of 120 mm Hg), both eyes were enucleated and bisected at the equator. The retinas then were dissected away under an operating microscope and were thoroughly dried in a Speed-Vac for 4 hours. After measurement of the retinal dry weight, the Evans Blue dye was extracted by incubating each retina in 200 mL of formamide (Sigma) for 18 hours at 70° C. The extract was then ultra-centrifuged (IEC Micromax RF) through Ultra free-MC tubes (30,000 NMWL Filter Unit, Millipore) at a speed of 6,000 rpm for 2 hours at 4° C.

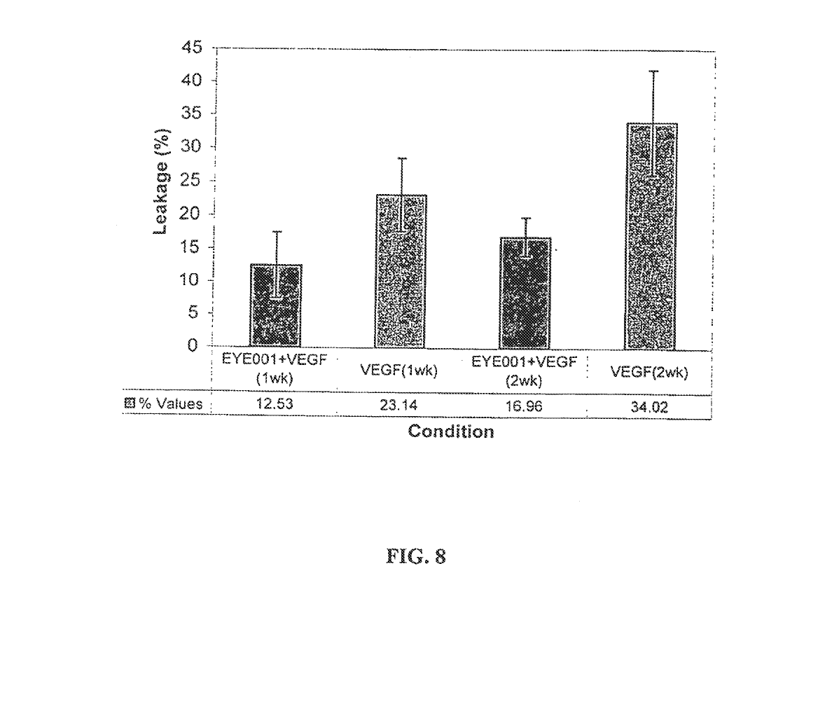

Sixty μL of the tissue-extracted Evans Blue dye supernatant and of the plasma-collected Evans Blue dye was used for triplicate spectrophotometric measurements. A background-subtracted absorbance was determined by measuring each sample at both 620 nm. (the absorbance maximum for Evans Blue dye) and 740 nm (the absorbance minimum for Evans Blue dye). The concentration of the dye in the extracts was calculated from a standard curve of Evans Blue dye in formamide. The results of these experiments are shown in FIG. 8.

Blood vessel leakage, as measured using Evans Blue dye release from blood vessels, was significantly reduced in eyes that were treated with the EYE-001 aptamer relative to control eyes that did not receive the aptamer. This reduction in blood vessel leakage was observed at all time points. While the % leakage of Evans Blue dye in the control eyes after one and two weeks was 23% and 34%, respectively, when the EYE-001 aptamer was administered transclerally, the % leakage of Evans Blue dye after one and two weeks was reduced to 12.5% and 17%, respectively. At both, the one and two week time points, the transcleral delivery of the EYE-001 aptamer reduced blood vessel leakage by about 50%.

These results demonstrate that the EYE-001 aptamer, when delivered transclerally, crossed the sclera and exerted at least one biological effect in vivo, i.e., reduced leakage from blood vessels within the eye.

### Example 3

### Implantable Mechanical Drug Delivery Device

In addition to the passive drug delivery devices described in Examples 1 and 2, the aptamer containing microspheres may be delivered to the ocular surface using a mechanical drug delivery device.

A mechanical device for delivering the anti-Vascular Endothelial Growth Factor aptamer (EYE001, formerly known as NX1838) (see, Drolet et al. (2000) PHARM. RES. 17:1503-1510; Ruckman et al. (1998) J. BIOL. CHEM. 273:20556-20567) can be fabricated in a device as shown in FIG. 1. The cavities, each having an internal volume of about 0.25 μL disposed about the surface of a titanium drum, are filled with the aptamer containing microspheres. The cavities then are sealed by coating the drum with parylene. A titanium overcoat then is applied onto the parylene layer by sputter deposition. The drum then is placed within a titanium casing having (i) a surface complementary in shape to the outer surface of an eye, (ii) an aperture in the surface to permit fluid to enter the casing and contact the outer surface of the drum, and (iii) a plurality of eyelets or fenestrations to permit the suturing of the device onto the outer surface of the eye.

The drum is placed within the casing in operative association with a power source, a magnetic drive mechanism, and a rotating puncturing member having a plurality of puncture needles disposed about a surface thereof. The magnetic drive mechanism is coupled to the drum via a biased ratchet mechanism, so that when the magnetic drive mechanism is periodically activated and deactivated, it incrementally rotates the drum. The drum also incrementally rotates the puncturing member via a gear mechanism preferably fabricated from interfitting titanium components. A needle disposed on the rotating puncturing member, when it contacts a cavity seal on the drug, pierces the seal to permit the release of aptamer out of the cavity. The needles on the puncturing member move in register with the cavities disposed about the surface of the incrementally rotating drum so that on a periodic basis a needle punctures the seal of a microsphere-containing cavity. Puncturing is repeated so that microspheres are sequentially released from a series of cavities to provide aptamer delivery over a prolonged period of time. The relative speed of rotation of the drum and puncturing member, and thus the rate of seal breakage, can he adjusted to change the rate of microsphere and, therefore, aptamer release.

### Example 4

### Implantation of Mechanical Drug Delivery Device

Surgical implantation of the mechanical drug delivery device of Example 3 can be performed under general or local anesthesia. In one approach, a 360-degree conjunctival peritomy is performed to open the conjunctiva and Tenon's capsule. Blunt scissors then are inserted into the quadrants between the rectus muscles, and the Tenon's capsule dissected from the underlying sclera. The rectus muscles then are isolated and looped on one or more retraction sutures, which permit rotation of the globe and exposure of the quadrants.

The device preferably is inserted into an accessible quadrant, for example, the superotemporal quadrant or the inferotemporal quadrant. Placement preferably is posterior to the muscle insertions and more preferably posterior to the equator. The device is placed temporarily in the selected quadrant to allow a determination of whether the conjunctiva and Tenon's capsule cover the device. If necessary, a relaxing incision may be made in the conjunctiva away from the quadrant selected for the device.

Fixation of the device may be accomplished using one or more of a tissue adhesive, scleral flaps, or sutures. Once the device is fixed to the sclera, the muscle retraction sutures are removed and the conjunctiva and Tenon's capsule closed over the device. The conjunctiva can then be sutured at the limbus using standard procedures. When implanted, the drug delivery device is activated to permit the microspheres to be administered to the surface of the eye at the desired rate.

### Incorporation By Reference

The entire disclosure of each of the publications and patent documents referred to herein is incorporated by reference in its entirety for all purposes to the same extent as if the teachings of each individual publication or patent document were included herein.

### Equivalents

The invention may be embodied in other specific forms without departing form the spirit or essential characteristics thereof. The foregoing embodiments, therefore, are to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A microsphere for sustained aptamer delivery, the microsphere comprising an anti-vascular endothelial growth factor aptamer and a biocompatible polymer.

2. The microsphere of claim 1, wherein the aptamer comprises from 0.1% (w/w) to 30% (w/w) of the microsphere.

3-4. (canceled)

5. The microsphere of claim 1, further comprising trehalose.

6. The microsphere of claim 5, wherein the mass ratio of aptamer to trehalose is at least 1:3.

7. The microsphere of claim 1, wherein the biocompatible polymer is a degradable polymer.

8. The microsphere of claim 7, wherein the degradable polymer is selected from the group consisting of polycarbonate, polyanhydride, polyamide, polyester, polyorthoester, and copolymers or mixtures thereof.

9-10. (canceled)

11. The microsphere of claim 7, wherein the polymer has a half-life of degradation under physiological conditions of at least 1 month.

12. The microsphere of claim 1, wherein the biocompatible polymer is a non-degradable polymer.

13. The microsphere of claim 12, wherein the non-degradable polymer is selected from the group consisting of polyether, vinyl polymer, polyurethane, cellulose-based polymer, and polysiloxane.

14-16. (canceled)

17. The microsphere of claim 1, wherein the microsphere has a diameter of about 15 μm.

18. A method of preventing, treating, or inhibiting an ocular disease in a mammal in need thereof, the method comprising administering to the mammal the microsphere of claim 1 in an amount sufficient to prevent, treat or inhibit the ocular disease.

19. The method of claim 18, wherein the administering step comprises contacting a scleral surface of the eye of the mammal with the microspheres.

20. The method of claim 18, wherein the administering step comprises administering the microspheres by intravitreal injection.

21. The method of claim 18, wherein the disease is optic disc neovascularization, iris neovascularization, retinal neovascularization, choroidal neovascularization, corneal neovascularization, vitreal neovascularization, glaucoma, pannus, pterygium, macular edema, vascular retinopathy, retinal degeneration, uveitis, inflammatory diseases of the retina, or proliferative vitreoretinopathy.

22-26. (canceled)

27. A method of treating age-related macular degeneration in a human, wherein the method comprises administering to a human in need thereof a microsphere formulation comprising an anti-VEGF aptamer.

28. The method of claim 27, wherein the microsphere formulation is administered locally.

29-30. (canceled)

31. The method of claim 27, wherein the aptamer is EYE001.

32. A method of preparing the microsphere of claim 1, the method comprising the steps of:
(a) dissolving a biocompatible polymer in a solvent to form a solution;
(b) combining the solution with an aptamer to produce a mixture; and
(c) combining the mixture of step (b) with a coacervating agent under conditions such that the biocompatible polymer forms microspheres containing the aptamer.

33. The method of claim 32, wherein step (b) further comprises the step of adding trehalose.

34. The method of claim 33, wherein the mass ratio of aptamer to the trehalose is at least 1:3.
